(19) United States
(12) Patent Application Publication
(10) Pub. No.: US 2014/0227794 A1
(43) Pub. Date: Aug. 14, 2014

(54) TIME SYNCHRONIZATION METHOD FOR DIAGNOSTIC STRIP ANALYSIS APPARATUS

(71) Applicants: In Hwan Choi, Suwon-si (KR); Jeong In Bag, Anyang Si (KR); Su Hwan Kim, Seoul (KR)

(72) Inventors: In Hwan Choi, Suwon-si (KR); Jeong In Bag, Anyang Si (KR); Su Hwan Kim, Seoul (KR)

(73) Assignee: PHILOSYS CO., LTD., Gunsan-si, Jeollabuk-do (KR)

(21) Appl. No.: 14/111,231

(22) PCT Filed: Apr. 28, 2011
(86) PCT No.: PCT/KR2011/003158

§ 371 Date: Mar. 4, 2014

(30) Foreign Application Priority Data
Apr. 13, 2011 (KR) 10-2011-0034100

(51) Int. Cl. G01N 33/66

(52) U.S. Cl. CPC G01N 33/66
USPC 436/95

(57) ABSTRACT

The present invention provides a time synchronization method for a diagnostic strip analysis apparatus. When a user sets time of the diagnostic strip analysis apparatus, time setting information is stored in a storage unit of the diagnostic strip analysis apparatus. Biological information is measured using the diagnostic strip analysis apparatus. The time setting information and measured amounts and measurement times of the biological information are simultaneously stored in the storage unit. The time setting information, the measured amounts and the measurement times, together with transmission times, are transmitted to a management medium. A control unit of the management medium determines whether time of the diagnostic strip analysis apparatus has been set. If the time of the diagnostic strip analysis apparatus has been set, the time setting information, the measured amounts, the measurement times, and the transmission times are stored in a storage unit of the management medium.

### TECHNICAL FIELD

The present invention relates, in general, to a time synchronization method for a diagnostic strip analysis apparatus and, more particularly, to a time synchronization method for a diagnostic strip analysis apparatus, which implements time synchronization between measurement time data stored in the diagnostic strip analysis apparatus and a management medium, thus efficiently managing data measured by the diagnostic strip analysis apparatus.

### BACKGROUND ART

Generally, a diagnostic strip analysis apparatus is a device for causing a biological sample such as blood to be absorbed into a test strip and analyzing information about blood glucose, pregnancy, virus infection, and an immunity level. Therefore, products such as a blood glucose meter or a pregnancy test strip have been mainly and widely used from the standpoint of the fact that the products can be conveniently used by users.

For example, a blood glucose meter is configured to check the concentration of glucose contained in the blood of an examinee and rapidly and easily monitor variations caused by instability in a blood glucose level. In particular, such a blood glucose meter is one of the important medical supporting instruments of examinees such as diabetics whose blood glucose must be checked several times a day.

In the prior art, there are the inconvenience of having to personally write blood glucose levels and measurement times on a blood glucose notebook after blood glucose has been measured using a blood glucose meter, and the inconvenience of having to always carry the blood glucose notebook on which the blood glucose levels are written when receiving medical treatment at a hospital. Further, there are a lot of difficulties in managing blood glucose.

However, recently, with the development of communication technology, blood glucose data measured by a blood glucose meter is transmitted to a management medium. The management medium systematically manages received blood glucose data, and provides services customized for individual persons, such as alimentotherapy, an exercise cure, prescription, and administration, on the basis of the received data, thus systematically and efficiently managing blood glucose.

Persons who use the management medium for biological information, such as blood glucose, periodically self-measure required biological information using test strips, and transmit measured data and measurement times to the management medium. Since the management medium must receive the measured data and the measurement times, analyze variations in biological data, and provide customized services suitable for the analyzed variations, the measured data and the measurement times received from the diagnostic strip analysis apparatus must be precisely recorded on the management medium, and the recorded times must be identical to the measurement times of the diagnostic strip analysis apparatus.

However, in many cases, users who use the diagnostic strip analysis apparatus do not manage the time of the diagnostic strip analysis apparatus; or the measurement times of the measured data which are transmitted from the diagnostic strip analysis apparatus are not individually managed by the management medium. Further, there are problems in that since it is realistically complicated to especially manage the measurement times of the diagnostic strip analysis apparatus in the management medium, it is difficult to manage the measured data of each user due to the discrepancy between the measurement times at which actual measurement is performed by the diagnostic strip analysis apparatus and times at which the management medium receives the measured data including the measurement times.

### DISCLOSURE OF INVENTION

### Technical Problem

Accordingly, the present invention has been made keeping in mind the above problems occurring in the prior art, and an object of the present invention is to provide a time synchronization method for a diagnostic strip analysis apparatus, which implements time synchronization between measurement time data stored in the diagnostic strip analysis apparatus and a management medium, thus efficiently managing data measured by the diagnostic strip analysis apparatus.

### Solution to Problem

In accordance with an aspect of the present invention, there is provided a time synchronization method for a diagnostic strip analysis apparatus, including a) when a user sets time of the diagnostic strip analysis apparatus, storing time setting information in a storage unit of the diagnostic strip analysis apparatus; b) measuring biological information using the diagnostic strip analysis apparatus; c) simultaneously storing the time setting information of the diagnostic strip analysis apparatus and measured amounts and measurement times of the biological information in the storage unit of the diagnostic strip analysis apparatus; d) transmitting the time setting information of the diagnostic strip analysis apparatus and the measured amounts and the measurement times of the biological information, together with transmission times, to a management medium; e) a control unit of the management medium recognizing the time setting information of the diagnostic strip analysis apparatus, and then determining whether time of the diagnostic strip analysis apparatus has been set; and f) if the control unit of the management medium determines that the time of the diagnostic strip analysis apparatus has been set, storing the time setting information of the diagnostic strip analysis apparatus and the measured amounts, the measurement times and the transmission times of the biological information, which have been transmitted to the management medium, in a storage unit of the management medium.

Preferably, the time synchronization method may further include, after f), g) transmitting information, which indicates that time synchronization has been implemented, to the diagnostic strip analysis apparatus.

Preferably, g) may be configured to transmit information about a difference between times set in the diagnostic strip analysis apparatus and the management medium based on time set in the management medium, together with information which indicates that time synchronization has been implemented, to the diagnostic strip analysis apparatus.

In accordance with another aspect of the present invention, there is provided a time synchronization method for a diagnostic strip analysis apparatus, including a) measuring biological information using the diagnostic strip analysis apparatus; b) simultaneously storing measured amounts and measurement times of the biological information of the diagnostic strip analysis apparatus, in a storage unit of the diagnostic strip analysis apparatus; c) transmitting the measured amounts and the measurement times of the biological information of the diagnostic strip analysis apparatus, together with transmission times, to a management medium; d) a control unit of the management medium recognizing time setting information of the diagnostic strip analysis apparatus, and then determining whether time of the diagnostic strip analysis apparatus has been set; and e) if the control unit of the management medium determines that the time of the diagnostic strip analysis apparatus has not been set, synchronizing the transmission times of the diagnostic strip analysis apparatus with time set in the management medium, inversely calculating measurement times of the biological information based on the transmission times, and then synchronizing pieces of time information of the management medium and the diagnostic strip analysis apparatus with each other.

Preferably, the time synchronization method may further include, after e), f) storing the measured amounts, measurement times and transmission times of the biological information of the diagnostic strip analysis apparatus, which have been time-synchronized, in the storage unit of the management medium, and transmitting time setting information indicating that time has been set to the diagnostic strip analysis apparatus; and g) storing the measured amounts, the measurement times and the transmission times of the biological information of the diagnostic strip analysis apparatus, which have been time-synchronized, together with the time setting information, in the storage unit of the diagnostic strip analysis apparatus.

Preferably, f) may further include transmitting information, which indicates that time synchronization has been implemented, to the diagnostic strip analysis apparatus.

### Advantageous Effects of Invention

In accordance with a time synchronization method for a diagnostic strip analysis apparatus according to the present invention, time synchronization is implemented between the diagnostic strip analysis apparatus and a measurement medium that receives data, including the measured amounts and the measurement times of biological information stored in the diagnostic strip analysis apparatus, so that measured data about biological information obtained by the diagnostic strip analysis apparatus can be precisely and efficiently managed, thus enabling the blood glucose and health condition of each user to be safely managed.

### BRIEF DESCRIPTION OF DRAWINGS

FIG. 1 is a diagram schematically showing the construction of a diagnostic strip analysis apparatus and a management medium according to the present invention;

FIG. 2 is a flowchart showing a time synchronization method for a diagnostic strip analysis apparatus according to an embodiment of the present invention; and

FIG. 3 is a flowchart showing a time synchronization method for a diagnostic strip analysis apparatus according to another embodiment of the present invention.

### BEST MODE FOR CARRYING OUT THE INVENTION

The above and other objects, features and advantages of the present invention will be more clearly understood from the following detailed description taken in conjunction with the accompanying drawings. Hereinafter, a time synchronization method for a diagnostic strip analysis apparatus according to the present invention will be described in detail with reference to the attached drawings.

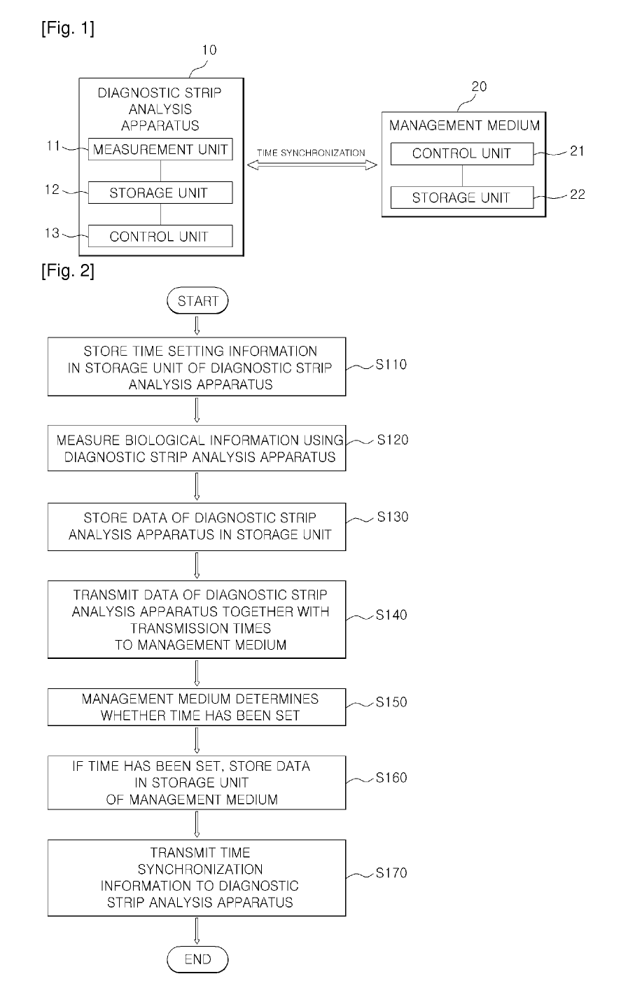

FIG. 1 is a diagram schematically showing the construction of a diagnostic strip analysis apparatus and a management medium according to the present invention.

As shown in FIG. 1, a diagnostic strip analysis apparatus 10 according to the present invention includes a measurement unit 11, a storage unit 12, and a control unit 13. The measurement unit 11 of the diagnostic strip analysis apparatus 10 is a component for measuring ingredients contained in a biological sample such as blood or urine, and mainly performs measurement in such a way that the biological sample is absorbed into or adsorbed to a test strip and then the test strip is inserted into the measurement unit 11 of the diagnostic strip analysis apparatus 10. The storage unit 12 of the diagnostic strip analysis apparatus 10 is a component for storing various types of data such as the measured amounts and measurement times of the biological information measured using the measurement unit 11. Further, the control unit 13 of the diagnostic strip analysis apparatus 10 is a component for executing commands related to various types of control and information processing such as the measurement, storage and transmission performed by the diagnostic strip analysis apparatus 10.

The management medium 20 according to the present invention includes a control unit 21 and a storage unit 22. The control unit 21 of the management medium 20 is a component for executing commands for receiving, processing and storing various types of data transmitted from the diagnostic strip analysis apparatus 10. Time synchronization is implemented between the diagnostic strip analysis apparatus 10 and the management medium 20 by the control unit 21 of the management medium 20. Further, the storage unit 22 of the management medium 20 is a component for storing various types of data transmitted from the diagnostic strip analysis apparatus 10 and various types of data processed by the control unit 21 of the management medium 20.

Therefore, even in cases where measurement time data corresponding to the measured amounts of biological information measured using the diagnostic strip analysis apparatus 10 indicates that the sequence of measurements is inconsistent, or where the measurement time data differs from that of the management medium because of several causes, such as the discharging of the diagnostic strip analysis apparatus 10 or the non-performance of time setting by the user, such cases can be easily solved by time synchronization between the diagnostic strip analysis apparatus 10 and the management medium 20 according to the present invention.

FIG. 2 is a flowchart showing a time synchronization method for a diagnostic strip analysis apparatus according to an embodiment of the present invention.

As shown in FIG. 2, the time synchronization method for the diagnostic strip analysis apparatus according to an embodiment of the present invention includes steps S110 to S160. At step S110, when a user sets the time of the diagnostic strip analysis apparatus, relevant time setting information is stored in the storage unit of the diagnostic strip analysis apparatus. At step S120, biological information is measured using the diagnostic strip analysis apparatus. At step S130, the time setting information of the diagnostic strip analysis apparatus, and the measured amounts and measurement times of the biological information are simultaneously stored in the storage unit of the diagnostic strip analysis apparatus. At step S140, the time setting information of the diagnostic strip analysis apparatus, and the measured amounts and the measurement times of the biological information, together with transmission times, are transmitted to the management medium. At step S150, the control unit of the management medium recognizes the time setting information of the diagnostic strip analysis apparatus and then determines whether the time of the diagnostic strip analysis apparatus has been set. If the control unit of the management medium determines that the time of the diagnostic strip analysis apparatus has been set, the time setting information of the diagnostic strip analysis apparatus, the measured amounts and the measurement times of the biological information, and the transmission times, which have been transmitted to the management medium, are stored in the storage unit of the management medium at step S160.

Hereinafter, the time synchronization method for the diagnostic strip analysis apparatus according to the embodiment of the present invention will be described in detail.

First, when the user sets the time of the diagnostic strip analysis apparatus, the time setting information is stored in the storage unit of the diagnostic strip analysis apparatus at step S110. Here, the time setting information is information indicating that time has been set, wherein when the measurement of a biological sample is performed, the time setting information is simultaneously stored and displayed together with the measured amount data and the measurement time of each piece of measured biological information. For example, the time setting information is displayed and stored in such a way that a flag or check mark is additionally provided to information in which the measured amounts and the measurement times are displayed. Thereafter, the biological information is measured using the diagnostic strip analysis apparatus at step S120. The measurement of the biological information is performed by inserting a test strip that absorbs the biological sample into the diagnostic strip analysis apparatus, as described above. Thereafter, the time setting information of the diagnostic strip analysis apparatus, and the measured amounts and the measurement times of the biological information are simultaneously stored in the storage unit of the diagnostic strip analysis apparatus at step S130. The control unit of the diagnostic strip analysis apparatus transmits the time setting information of the diagnostic strip analysis apparatus and the measured amounts and the measurement times of the biological information, together with transmission times, to the management medium at step S140. Thereafter, the control unit of the management medium recognizes the time setting information of the diagnostic strip analysis apparatus and then determines whether the time has been set at step S150. Here, since the time setting information of the diagnostic strip analysis apparatus has been transmitted to the management medium, the control unit of the management medium determines whether time has been set by checking whether the time setting information of the diagnostic strip analysis apparatus is present. Thereafter, if the control unit of the management medium determines that the time of the diagnostic strip analysis apparatus has been set, separate time synchronization is not required in general because the measured amounts of biological information have been sequentially obtained by the diagnostic strip analysis apparatus according to the time sequence, and thus the time setting information of the diagnostic strip analysis apparatus, the measured amounts, the measurement times, and the transmission times of the biological information, which have been transmitted to the management medium, are stored in the storage unit of the management medium at step S160. In this way, time synchronization between the diagnostic strip analysis apparatus and the management medium is completed.

The time synchronization method for the diagnostic strip analysis apparatus according to the embodiment of the present invention may further include, after step S160, the step S170 of allowing the management medium to transmit information indicating that time synchronization has been implemented to the diagnostic strip analysis apparatus, thus allowing the user to recognize the information about time synchronization. Further, when a time difference occurs between the diagnostic strip analysis apparatus and the management medium, the management medium transmits information about the difference between the set times of the diagnostic strip analysis apparatus and the management medium on the basis of the time set in the management medium, together with information indicating that time synchronization has been implemented, to the diagnostic strip analysis apparatus, and thus the user can subsequently cause the times of the management medium and diagnostic strip analysis apparatus to be identical to each other if necessary.

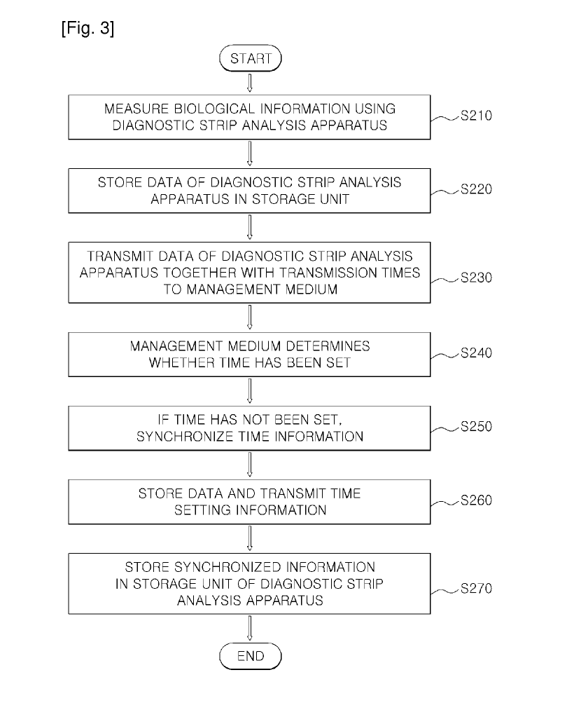

FIG. 3 is a flowchart showing a time synchronization method for a diagnostic strip analysis apparatus according to another embodiment of the present invention.

As shown in FIG. 3, when time is not set before a biological sample is measured using the diagnostic strip analysis apparatus, or when the diagnostic strip analysis apparatus is just purchased and immediately used, time setting information is not stored in and displayed on the storage unit of the diagnostic strip analysis apparatus. In this case, biological information is measured using the diagnostic strip analysis apparatus at step S210. After the measured amounts and the measurement times of the biological information measured using the diagnostic strip analysis apparatus have been simultaneously stored in the storage unit of the diagnostic strip analysis apparatus at step S220, the diagnostic strip analysis apparatus transmits the measured amounts and the measurement times of the biological information measured using the diagnostic strip analysis apparatus, together with transmission times, to the management medium at step S230. The control unit of the management medium determines whether the time of the diagnostic strip analysis apparatus has been set by recognizing the time setting information of the diagnostic strip analysis apparatus among the pieces of transmitted data at step S240. In this case, since the diagnostic strip analysis apparatus did not store and transmit time setting information, the control unit of the management medium determines that the time of the diagnostic strip analysis apparatus has not been set, and synchronizes the transmission times of the diagnostic strip analysis apparatus with time set in the management medium, inversely calculates the measurement times of the biological information based on the transmission times, and then synchronizes pieces of time information of the management medium and the diagnostic strip analysis apparatus with each other at step S250. In this way, when the time setting information is not stored in the diagnostic strip analysis apparatus, time synchronization between the diagnostic strip analysis apparatus and the management medium is implemented based on the transmission times stored in the management medium and the measurement times calculated by the management medium.

Thereafter, the time synchronization method of the present invention may further include steps S260 and S270. At step S260, the control unit of the management medium stores the measured amounts, the measurement times, and the transmission times of the biological information of the diagnostic strip analysis apparatus, which have been time-synchronized, in the storage unit of the management medium, and transmits time setting information indicating that time has been set to the diagnostic strip analysis apparatus. Further, at step S270, the measured amounts, the measurement times, and the transmission times of the biological information of the diagnostic strip analysis apparatus, which have been time-synchronized, together with the time setting information, are stored in the storage unit of the diagnostic strip analysis apparatus. Further, step S260 may further include the step of transmitting information, indicating that time synchronization between the diagnostic strip analysis apparatus and the management medium has been implemented, to the diagnostic strip analysis apparatus.

The time synchronization method for the diagnostic strip analysis apparatus according to the present invention can be usefully utilized when time is not set before the user uses the diagnostic strip analysis apparatus, or when the times of the diagnostic strip analysis apparatus and the management medium differ from each other. However, the present invention can also be utilized in the case where discharging suddenly occurs and then the set time is changed while a plurality of pieces of biological information are measured using the diagnostic strip analysis apparatus.

For example, it is assumed that after the user has set the time, measurement is performed three times, and values are measured every two hours as follows:

1. 9:00 on Apr. 5, 2011 100 mg/dl √

2. 11:00 on Apr. 5, 2011 90 mg/dl √

3. 13:00 on Apr. 5, 2011 120 mg/dl √

In this case, three pieces of data at 1 to 3 are marked “√”, indicating that data was measured after time setting, and are then stored. In this case, when the user replaces a battery, the diagnostic strip analysis apparatus is completely discharged, and the information about the time setting of the diagnostic strip analysis apparatus is initialized, for example, to Jan. 1, 2010. However, when the user does not set the time through carelessness and continues to measure biological information, values are assumed to be measured as follows:

4. 1:00 on Jan. 1, 2010 80 mg/ml √

5. 3:00 on Jan. 1, 2010 150 mg/ml √

In this case, two pieces of data at 4 and 5 do not have the time setting mark “√”. Therefore, pieces of data about the measured amounts and the measurement times of the biological information, stored in the diagnostic strip analysis apparatus, are transmitted to the management medium in the state in which time setting information is stored in the pieces of data at 1 to 3 and is not stored in the pieces of data at 4 and 5. In this case, since the management medium may determine that time setting has been performed on the data at 1 to 3, there is no great problem. However, since the management medium may not determine that time setting has been performed on the data at 4 and 5, time synchronization with the diagnostic strip analysis apparatus is primarily performed based on the transmission times. Therefore, after the transmission times have been synchronized, inverse calculation is performed based on the transmission times, and thus time synchronization with the data at 4 and 5 is performed. That is, the times of the pieces of data at 4 and 5 are synchronized with the actual measurement times, as follows:

4. 15:00 on Apr. 5, 2011 80 mg/dl √

5. 17:00 on Apr. 5, 2011 150 mg/dl √

In this way, the time synchronization method for the diagnostic strip analysis apparatus according to the present invention is advantageous in that even if the user does not set time during a procedure for using the diagnostic strip analysis apparatus, pieces of data about the measurement of biological information can be sequentially managed, thus improving the convenience of patients and a manager who use the diagnostic strip analysis apparatus.

Although the preferred embodiments of the present invention have been disclosed for illustrative purposes, the present invention is not limited to the above-described specific embodiments. That is, those skilled in the art will appreciate that various modifications and changes are possible, without departing from the spirit and scope of the invention as disclosed in the accompanying claims, and all suitable modifications and changes and equivalents thereof should be interpreted as being included in the scope of the present invention.

### INDUSTRIAL APPLICABILITY

According to the present invention, a time synchronization method for a diagnostic strip analysis apparatus is advantageous in that time synchronization is implemented between the diagnostic strip analysis apparatus and a measurement medium that receives data, including the measured amounts and the measurement times of biological information stored in the diagnostic strip analysis apparatus, so that measured data about biological information obtained by the diagnostic strip analysis apparatus can be precisely and efficiently managed, thus enabling the blood glucose and health condition of each user to be safely managed.

1. A time synchronization method for a diagnostic strip analysis apparatus, comprising:
a) when a user sets time of the diagnostic strip analysis apparatus, storing time setting information in a storage unit of the diagnostic strip analysis apparatus;
b) measuring biological information using the diagnostic strip analysis apparatus;
c) simultaneously storing the time setting information of the diagnostic strip analysis apparatus and measured amounts and measurement times of the biological information in the storage unit of the diagnostic strip analysis apparatus;
d) transmitting the time setting information of the diagnostic strip analysis apparatus and the measured amounts and the measurement times of the biological information, together with transmission times, to a management medium;
e) a control unit of the management medium recognizing the time setting information of the diagnostic strip analysis apparatus, and then determining whether time of the diagnostic strip analysis apparatus has been set; and
f) if the control unit of the management medium determines that the time of the diagnostic strip analysis apparatus has been set, storing the time setting information of the diagnostic strip analysis apparatus and the measured amounts, the measurement times and the transmission times of the biological information, which have been transmitted to the management medium, in a storage unit of the management medium.

2. The time synchronization method according to claim 1, further comprising, after f), g) transmitting information, which indicates that time synchronization has been implemented, to the diagnostic strip analysis apparatus.

3. The time synchronization method according to claim 2, wherein g) is configured to transmit information about a difference between times set in the diagnostic strip analysis apparatus and the management medium based on time set in the management medium, together with information which indicates that time synchronization has been implemented, to the diagnostic strip analysis apparatus.

4. A time synchronization method for a diagnostic strip analysis apparatus, comprising:
a) measuring biological information using the diagnostic strip analysis apparatus;
b) simultaneously storing measured amounts and measurement times of the biological information of the diagnostic strip analysis apparatus, in a storage unit of the diagnostic strip analysis apparatus;
c) transmitting the measured amounts and the measurement times of the biological information of the diagnostic strip analysis apparatus, together with transmission times, to a management medium;
d) a control unit of the management medium recognizing time setting information of the diagnostic strip analysis apparatus, and then determining whether time of the diagnostic strip analysis apparatus has been set; and
e) if the control unit of the management medium determines that the time of the diagnostic strip analysis apparatus has not been set, synchronizing the transmission times of the diagnostic strip analysis apparatus with time set in the management medium, inversely calculating measurement times of the biological information based on the transmission times, and then synchronizing pieces of time information of the management medium and the diagnostic strip analysis apparatus with each other.

5. The time synchronization method according to claim 4, further comprising, after e):
f) storing the measured amounts, measurement times and transmission times of the biological information of the diagnostic strip analysis apparatus, which have been time-synchronized, in the storage unit of the management medium, and transmitting time setting information indicating that time has been set to the diagnostic strip analysis apparatus; and
g) storing the measured amounts, the measurement times and the transmission times of the biological information of the diagnostic strip analysis apparatus, which have been time-synchronized, together with the time setting information, in the storage unit of the diagnostic strip analysis apparatus.

6. The time synchronization method according to claim 5, wherein f) further comprises transmitting information, which indicates that time synchronization has been implemented, to the diagnostic strip analysis apparatus.
